(19) United States
(12) Patent Application Publication
(10) Pub. No.: US 2003/0099657 A1
(43) Pub. Date: May 29, 2003

(54) Drug-heteropolymers

(76) Inventor: Gary Israel Azoff, Athens, GA (US)

Correspondence Address: Gary Israel Azoff, P.O. Box 82148, Athens, GA, 30608, US

(21) Appl. No.: 09/991,798

(22) Filed: Nov. 26, 2001

(51) Int. Cl. A61K 48/00; A61K 39/395; C07K 16/46

(52) U.S. Cl. 424/178.1; 514/44; 530/391.1

(57) ABSTRACT

It has long been recognized that effective delivery systems are preferred for chemotherapeutic agents. The present invention addresses this need. First, chemotherapeutic agents are attached to monoclonal antibodies; second, the antibody is combined with DNA to yield a stable heteropolymer; and, third, the drug-heteropolymer is injected in a therapeutically effective amount.

The present invention is addressed specifically to cancer and rheumatic diseases. Heteropolymers have been used effectively in lupus-like models, but drugs have never been attached. If they were, it could be another step towards more complete treatments. In cancer, drugs have been attached to monoclonal antibodies with disappointing results, because the body turns against the unmasked antibody, sometimes making it antigenic itself. DNA is a stable substance in cancer which does not elicit antibodies, and, in lupus, has been shown to be an aid to administering monoclonal antibodies.

The methods of producing DNA-antibody polymers and attaching drugs to monoclonal antibodies are well known and established; the invention is easily and inexpensively carried out.

Conjunction of Drug with antibody (Example drugs: Doxorobucin) (Pruam, et al: Cancer Research 55, 2353-56, June 1995).

The antibody is reduced with DTT (trichlorothane) at room temperature. After 30 minutes, the mixture is passed through an RD-10 (Pharmacia) column to remove the unreacted DTT. Doxorubicine (DOX) is treated with glutaraldehyde at room temperature and added to the antibody. The mixture is mixed in a rotary shaker for 1 hour. Unreacted DOX is removed by gel PD-10-filtration.

Biotinylated double stranded DNA is cross-linked to the antibody by incubation for 30 minutes at room temperature with 48 ml borate saline. (Ross, G. D. et al: Arthritis and Rheumatism 2005-14, 1985).

I claim the process comprising the following steps:

1. A chemotherapeutic agent attached to a monoclonal antibody. The antibody is combined with DNA to yield a heteropolymer. The drug heteropolymer is injected in a therapeutically effective amount.

2. The process of claim 1 for cancer.

3. The process of claim 1 for rheumatic disease.

4. A method of attaching a chemotherapeutic agent with a monoclonal antibody, combining the antibody with DNA to yield a drug-heteropolymer, and injecting the drug-heteropolymer in a therapeutically effective amount.

5. The process of claim 4 for cancer.

6. The process of claim 4 for rheumatic disease.
